(19) United States
(12) Patent Application Publication
(10) Pub. No.: US 2008/0103445 A1
(43) Pub. Date: May 1, 2008

(54) Method and Apparatus for Detecting Air Bubbles

(76) Inventors: David H. Blaine, Salt Lake City, UT (US); Mark D. Stringham, Salt Lake City, UT (US); Frank A. Crandall, Salt Lake City, UT (US); Terral Bart Michaelson, Woods Cross, UT (US); Philip N. Eggers, Salt Lake City, UT (US); Timothy A. Riley, Salt Lake City, UT (US); Shawn P. Fojtik, Park City, UT (US); Allan E. Smith, Salt Lake City, UT (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(21) Appl. No.: 11/862,155

(22) Filed: Sep. 26, 2007

Related U.S. Application Data
(60) Provisional application No. 60/827,622, filed on Sep. 29, 2006.

(51) Int. Cl. A61M 1/00; G01F 13/00; A61M 39/00; F04B 49/00; G01L 7/00

(52) U.S. Cl. 604/122; 73/861.41; 604/533; 417/278; 73/700; 417/300

(57) ABSTRACT

An air bubble detection system includes an air bubble detector which is disposed downstream from a syringe or manifold used during an angiography procedure. The air bubble detection system monitors contrast medium being injected into the patient and creates an alarm or other indication if the air bubble exceeds any desired threshold.

### RELATED APPLICATIONS

The present application claims the benefit of U.S. Provisional Application Ser. No. 60/827,622, filed Sep. 29, 2006, which is expressly incorporated herein in its entirety.

### BACKGROUND OF THE INVENTION

1. The Field of the Invention

The present invention relates to a method and apparatus for detecting air bubbles while injecting fluid into a patient. More specifically, the present invention relates to apparatuses and methods for detecting the introduction of air downstream from an injection port during use of a catheter in an invasive procedure, so as to minimize the risk of an air embolus being introduced into the bloodstream of a patient.

2. State of the Art

There are a variety of medical procedures in which a liquid is injected into a patient. In some situations, the introduction of air into the patient may be of little if any concern. In other procedures, however, the introduction of air can be of significant concern. One procedure in which the introduction of air can literally create a life or death scenario is angiography.

The use of angiography for a variety of medical procedures has become well known. In angiography a catheter is advanced into the circulatory system of an individual. A contrast medium is then injected into the individual through the catheter. The contrast medium is observed using a fluoroscope so as to allow physicians to better view structures inside the body. Angiography can be used simply to assist the physician with advancement of the catheter, with the imaging being used to help the physician navigate through the arteries, etc. It may also be used in procedures such as the placement of a stent in a venous system so as to prevent occlusion or collapse of a blood vessel to ensure that the stent is placed at the proper location to prevent closing of the vessel. Likewise angiography can be used to take pictures of heart valves or other structures as they function in order to determine the proper treatment for circulatory problems. Angiography also can be used to determine the location of a blood clot in the brain so that ameliorative therapies may be focused on the particular point of concern.

An angiography can be a complex procedure. Typically a surgeon will utilize one or more syringes to inject the contrast medium through the catheter. Thus, as shown in FIG. 1A, a syringe 10 may be attached to a catheter 14 via a luer lock 16 or other attachment mechanism. The catheter 14 includes one or more lumens (not shown), at least one of which exits at or adjacent a distal end 14a of the catheter. (In some procedures, a multi-lumen catheter may be used to inject contrast medium at a plurality of locations. Thus, the catheter may have an exit port for one lumen at the distal tip, and another several inches back. In such a situation, the catheter would typically include luer locks for two or more syringes so that contrast medium could be released selectively from either exit port.)

While the configuration shown in FIG. 1A is advantageous for some simple procedures, there are many procedures in which larger quantities of contrast medium will be used. For example, when viewing the functioning of a heart valve, it can be necessary to inject large amounts of contrast medium in a short amount of time to ensure proper visualization of the valves' movement as the heart expands and contracts.

During the procedure the physician will typically be watching a fluoroscope or other monitor to view the interior structures of the body made visible by the contrast medium. While he or she does so, the physician may be manipulating one or more plungers to inject a desired amount of contrast medium to aid in that visualization. Because a physician must carefully watch the monitor, the physician will typically not be looking at the syringes as they are used to inject fluid through the catheter.

FIG. 1B shows a contrast medium injection system, generally indicated at 20, as may be used in a cardioangiography. The catheter 14 is connected to a manifold 24 which receives three syringes 28. The manifold 24 also is connected to a contrast medium infusion line 32 which is connected to a contrast medium pump 36. A portion of the contrast medium infusion line 32 and the contrast medium pump 36 may be disposed outside of the sterile field, represented by curved line 40. To control the contrast medium pump 36, a switch 44 may be provided to allow the physician to activate or deactivate the pump with either the hand or foot. Thus, though the contrast medium pump 36 is typically disposed outside the sterile field 40, it can be actuated from within the sterile field.

The catheter 14 will typically be inserted into the femoral artery and advanced toward the heart. As the physician advances the catheter, the location of the distal end 14a can be monitored by injecting small amounts of contrast medium and watching the monitor 48 of the fluoroscope. Once the distal end 14a of the catheter 14 is in the desired position, the syringes 28 can be used to inject larger amounts of contrast medium, or the pump 36 can be used to inject a large amount of contrast medium in a very short amount of time.

One concern when conducting an angiography is the introduction of air into the circulatory system of the patient. Because angiography often takes place near the heart and/or the brain, the introduction of air can run a substantial risk of creating an embolism and causing serious injury to the patient. Thus, it is important that the contrast medium injecting system be free from any substantial quantity of air. In some procedures, the amount of air should not exceed 50 μl (microliters).

To accomplish this goal, the surgical staff will typically fill each of the syringes with contrast medium and attempt to remove any air pockets. The syringes are then typically attached to a manifold which connects to the catheter, and the manifold and catheter are primed to remove excess air.

While surgical staff is usually diligent in removing the air, there remains a risk for the patient. If the syringes, manifold, and catheter, have not been carefully primed and tightly sealed, there remains a risk that air can remain in the contrast medium flow path or be drawn in through the attachments between the syringes and manifold and/or manifold and catheter. Additionally, if pressure is applied to the syringes, the syringe may contain a larger quantity of air that has been compressed by the pressure, thus leading to the false assumption that only a small bubble of air is present.

In recent years some have appreciated the concern of large quantities of air being introduced into a patient and have placed air bubble detectors adjacent to the contrast medium pump 36 (FIG. 1B). However, such sensors are substantially upstream from the manifold and other potential points of failure and provide no protection against air inadvertently administered through the syringe or manifold connections. Thus, there is a need for an improved mechanism for preventing air embolisms when injecting a fluid into a patient.

### OBJECTS AND SUMMARY OF THE INVENTION

It is an object of the present invention to provide an apparatus and method for reducing the risk of air or other gas bubbles being introduced into a patient.

In accordance with one aspect of the invention, an air bubble detector is disposed distally (i.e. downstream or toward the patient) of the point(s) of fluid injection, through which fluid is injected into a patient.

In accordance with one aspect of the invention the air bubble detector is disposed so that at least the air bubble sensor is disposed downstream from the syringe in order to detect any air that may be introduced into the catheter by actuation of the syringe.

In accordance with another aspect of the present invention, the air bubble detector is disposed downstream from the manifold which receives an injected fluid from a plurality of syringes so as to detect a predetermined amount of air passing from the manifold into the catheter regardless of whether the air enters the manifold from the syringes, from connections of the syringes to the manifold, or from a position upstream from the manifold.

In accordance with another aspect of the invention, the air bubble detector may be configured such that part of the air bubble detector is disposed within the sterile field and a portion of the bubble detector may be disposed outside of the sterile field so as to minimize the portions of the bubble detector that must be either sterilized or disposed of following a procedure.

In accordance with another aspect of the present invention, the air bubble detector is configured for attachment between the manifold and the catheter so as to form part of the flow path of the contrast medium injection system.

In accordance with another aspect of the invention, the air bubble detector may be configured for placement on the catheter so as to detect the presence of air bubbles within the catheter.

These and other aspects of the present invention are realized in an air bubble detector and in an injection system as shown and described in the following figures and related description.

### BRIEF DESCRIPTION OF THE DRAWINGS

Various embodiments of the present invention are shown and described in reference to the numbered drawings wherein:

FIG. 1A shows a syringe and catheter in accordance with the principles of the prior art;

FIG. 1B shows a schematic of a fluid injection system for use in a medical procedure, such as angiography, in accordance with the principles of the prior art;

FIG. 2 shows a schematic of a fluid injection system for a medical procedure in accordance with the principles of the present invention;

FIG. 3 shows a close-up side view of an air bubble detector sensor disposed along the a fluid infusion line;

FIG. 3A shows a close-up, top view of the air bubble detector sensor shown in FIG. 3;

FIG. 3B shows a manifold configured to receive an air bubble detector sensor as shown in FIGS. 3 and 3A;

FIG. 3C shows an alternate configuration of a manifold and air bubble detector as may be used with the air bubble detector shown in FIGS. 3 and 3A;

FIG. 4 shows a top, fragmented view of a fluid injection system formed in accordance with the principles of the present invention;

FIG. 4A shows an air bubble detector sensor and a monitor in accordance with the principles of the present invention;

FIG. 4B shows an alternate embodiment of an air bubble detector in accordance with the present invention;

FIG. 4C shows yet another embodiment of an air bubble detector in accordance with the present invention;

FIG. 5 shows an alternate embodiment of an air bubble detection system in accordance with the principles of the present invention;

FIG. 6 shows yet another embodiment of an air bubble detection system in accordance with the principle of the present invention;

FIG. 7 shows the air bubble detector of FIG. 4B in use with a single syringe/catheter system;

FIG. 8 shows a combined manifold and air bubble detector sensor in accordance with the present invention;

FIG. 9 shows a combined pressure sensor and air bubble detector in accordance with the principle of the present invention; and

FIG. 10 shows a multifunction inline sensor in accordance with the principles of the present invention.

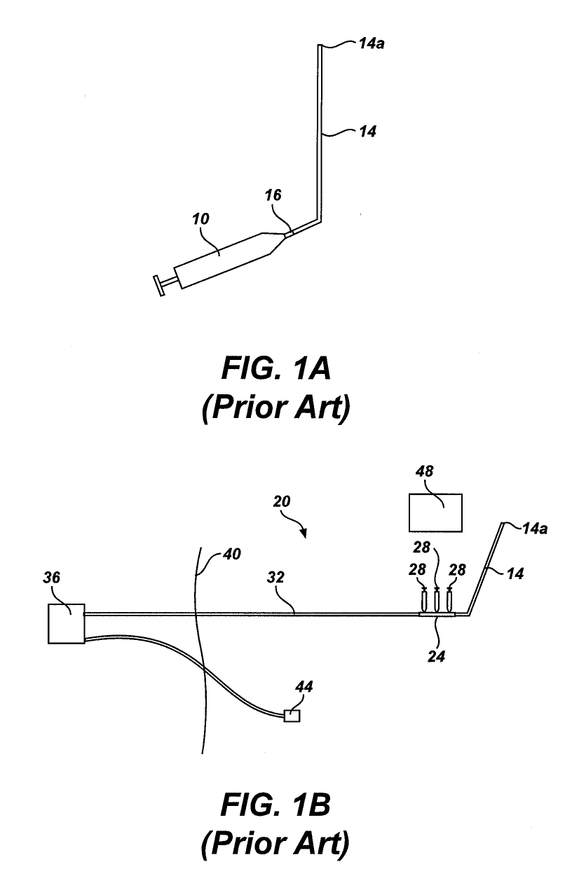

It will be appreciated that the drawings are illustrative and not limiting of the scope of the invention which is defined by the appended claims. It is further appreciated that not all aspects or structures of the invention may be shown in a single drawing, and as such various drawings illustrate smaller parts of the invention shown in other drawings. The various embodiments shown accomplish various aspects and objects of the invention, and it is not necessary that any particular embodiment accomplish all aspects and objects of the invention.

### DETAILED DESCRIPTION

The drawings will now be discussed in reference to the numerals provided therein so as to enable one skilled in the art to practice the present invention. The drawings and descriptions are exemplary of various aspects of the invention and are not intended to narrow the scope of the appended claims. It is appreciated that not all structures and elements of the invention may be shown in a single drawing and multiple drawings are therefore presented, each drawing more clearly illustrating all or a portion of the invention, and various parts of the drawings may be combined with parts of other drawings. Furthermore, it will be appreciated that various portions of the invention will be discussed with respect to various figures and may not be repeated with respect to each figure to provide a clear and concise disclosure of the invention. Additionally, it will be appreciated that not all embodiments or ports of embodiments need meet each object of the invention and such should not be viewed as limiting the appended claims.

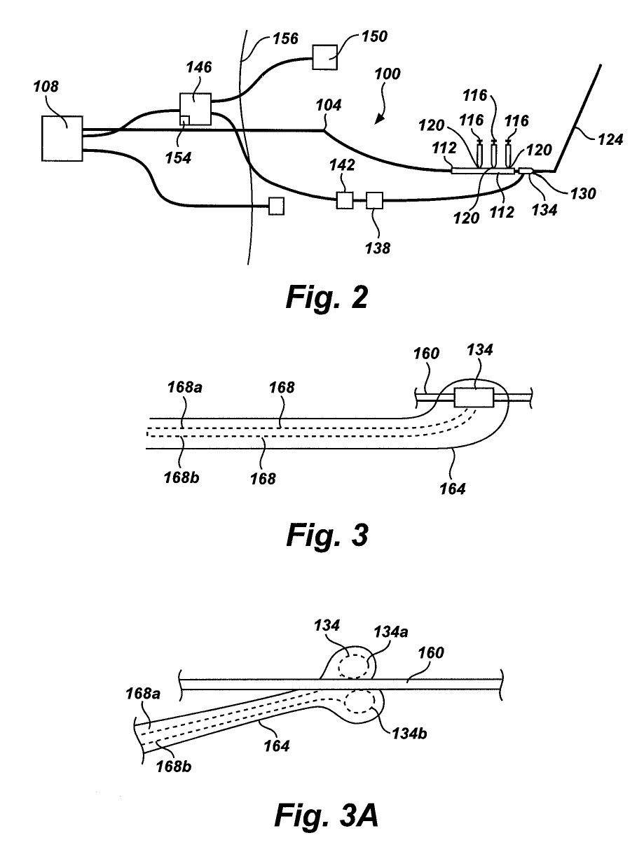

Turning now to FIG. 2, a schematic diagram is shown of a fluid injection system in accordance with the present invention which will be discussed in the context of injecting contrast medium in an angiography procedure. It will be appreciated that like systems could be used for other procedures as well.

The fluid injection system, generally indicated at 100, includes an infusion line 104 for carrying contrast medium from a contrast medium pump 108 to the manifold 112. A plurality of syringes 116 are attached to a plurality of fluid injection ports 120 on the manifold 112. Each of the syringes 116 and each of the fluid injection ports 120 raises a risk that air will be injected or drawn into the fluid infusion path as it extends to the catheter 124. (While the catheter is shown herein as being a straight member, it will be appreciated that the catheters are usually flexible to accommodate the tortuous structure of the circulatory system.)

In accordance with the present invention, an air bubble detector 130 is disposed at least partially downstream from the contrast medium injection ports 120 on the manifold. As will be explained in additional detail below, this allows for the detection of air (or other gas—collectively referred to herein as air) bubbles regardless of whether they have been introduced from the pump 108, the syringes 116, the fluid injection ports 120 or the attachment points of the manifold 112.

The air bubble detector 130 may include several parts. First, the air bubble detector 130 typically includes a sensor 134. A variety of different sensors may be used. Two commonly used types of sensors for bubble detection are ultrasonic sensors and optical sensors. Ultrasonic sensors typically use a piezoelectric material, such as a crystal, PVDF or ceramic material to generate high frequency sound (ultrasound). The sound waves travel through the conduit which is being monitored for air bubbles. If the conduit is filled with liquid, the ultrasonic signals are received by a piezoelectric material on the opposite side. The piezoelectric material creates an electrical signal in response to the sound waves, thereby indicating that liquid is present in the tube.

One ultrasonic bubble detector sold by ZEVEX, Inc. of Salt Lake City, Utah is particularly advantageous, as the frequency of the electrical signal driving the piezoelectric material is swept. Sweeping the frequency provides a more reliable result and reduces false positives created by mismatched transmitter/receiver pairs, environmental conditions, etc. This is due to the fact that the combined resonance of the sensor, the sensor housing, tubing, fluid, etc., can vary due to size differences, material differences, temperature and other environmental conditions. Thus, the combined or system resonance can change for a variety of reasons which cannot be readily controlled either during production of the sensor or during use by the physician. By using the circuitry to sweep the frequency, changes in the size of the manifold or housing, the conduit, the temperature, etc., can be adapted to without requiring input or adjustment by the user.

Air bubble detectors can also take other forms. For example, an optical emitter can direct light into a conduit configured for carrying fluids. If air is present, the light may be directed to a receiver, while the presence of an opaque liquid will substantially prevent the transmission of light. If clear liquids are used, the presence of air will cause a different index of refraction to be present at the chamber/fluid interface than if a clear liquid is present. A more detailed explanation of optical bubble detectors is set forth in U.S. Pat. No. 6,531,708, which is expressly incorporated herein. Those skilled in the art will appreciate that there are numerous different methods for determining the presence of air which could be used. For brevity, the sensor will be discussed in the context of an ultrasonic sensor. (It will be appreciated, however, that the claims should not be limited to such unless specifically limited therein).

The air bubble detector 130 may also include a signal processing unit 138 disposed in communication with the sensor 134. The signal processing unit 138 receives input from the sensor 134 to generate signals determinative of whether a bubble is present. The signal processing unit 138 can use buffers or other means for reducing noise in the signals.

The air bubble detector 130 will also typically include an indication or alarm mechanism 142 for providing an indication that an air bubble is present in the contrast medium infusion path. The indication mechanism may include a display panel for indicating status. It may also include a mechanism for producing a human perceptible alarm, such as a light which can be made to flash, and/or a speaker for emitting an alarm signal when a bubble has been detected.

The air bubble detector 130 can also include a communication module 146. The communication module 146 can be used for communications within the air bubble detector, or which other structures such as a contrast medium pump 108 or a fluoroscope monitor 150. The communications module 146 may be wired, or may rely on wireless technologies including, but not limited to, radio frequency, BLUETOOTH, and other wireless protocols.

The air bubble detector 130 may also include an injection control mechanism 154. The injection control mechanism 154 may be disposed in communication with the contrast medium pump 108 to automatically stop injection if an air bubble of sufficient size is detected. It will be appreciated that, especially under high pressure injection, the air bubble detector 130 could stop the pump 108 much more quickly than the physician's reflexes. In the alternative, the injection control mechanism 154 could temporarily suspend injection of contrast medium subject to an override by the physician.

While the various potential components of the air bubble detector 130 are discussed and shown as separate structures in FIG. 2, it will be appreciated by one of ordinary skill in the art that some or all of the components of the air bubble detector could be integrated into a single unit. Additionally, some components may serve multiple functions, or the functions described for the various components could be performed by separate structures.

Those skilled in the art will appreciate that such a system provides a significant improvement in safety. In the event that the syringes 116, the manifold 112 and the catheter 124 have not been checked and primed properly, the air bubble detector 130 will allow the physician to detect and prevent an air bubble from passing through the catheter and into the patient.

One challenge which is present in the context of an angiography, is use of the air bubble detector 130 within the sterile field, indicated by curved line 156. Because ultrasonic air bubble detectors can be relatively expensive, provision can be made to either facilitate reuse of at least part of the air bubble detector, or to keep the expense of the air bubble detector to a minimum when practical. As is discussed in detail below, there are several methods by which this can be accomplished.

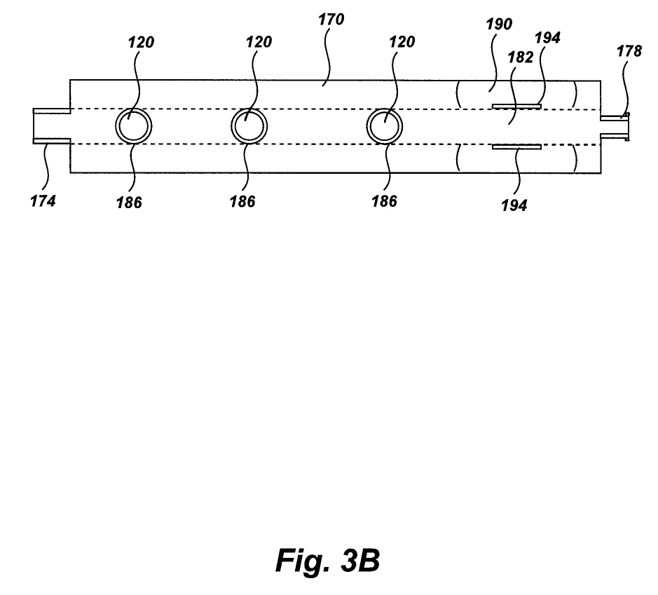

Turning now to FIG. 3, there is shown a close up view of the air bubble detector sensor 134. The sensor 134 is placed along the contrast medium path, represented by conduit 160, within the sterile field. The contrast medium path could be a portion of the manifold 112, a line leading from manifold to the catheter 124, or the catheter itself. To enable the sensor 134 to be reused for a number of procedures without repeatedly sterilizing the sensor, the sensor is disposed inside of a sheath 164. The sheath 164 is preferably long enough to cover the sensor 134 and associated wires 168 to a position outside the sterile field, or at least for a distance several feet from the patient. Thus, it is unlikely that the sensor 134 and associated wires 168 will ever come into contact with body fluids from the patient or other potential contaminants. For safety, they may be swabbed with alcohol if they are intended to be reused.

FIG. 3A shows a top view of the sensor 134 disposed about the conduit 160. In use, an electrical signal is sent down one wire 168a to the transmitter portion 134a of the sensor 134. The transmitter portion 134a of the sensor 134 generates an ultrasonic signal which is transmitted into the conduit 160. Those skilled in the art will appreciate that the transmission of the signal may occur via a dry coupling through the sheath 164, or coupling gels may be used. The ultrasonic signal is received or not by the receiver portion 134b which generates an electrical signal responsive to the ultrasonic signal received and passes the electrical signal along the wire 168b. The strength of the signal correlates to whether air is present in the conduit 160. Thus, a full signal indicates the lack of air in the conduit. A weaker than normal signal may indicate the presence of a small bubble, while no signal may indicate a large bubble, or a failure of the sensor. By monitoring the strength of the signal, the air bubble detector can indicate whether a bubble is present, or only generate an alarm when the bubble exceeds some predetermined threshold.

Turning now to FIG. 3B, there is shown a manifold 170 formed in accordance with the principles of the present invention. The manifold 170 includes a first connector 174, such as a female luer lock connector, at one end, and a second connector 178, such as a male luer lock connector. The connectors 174 and 178 allow the manifold 170 to be attached to a contrast medium supply line and to a catheter as is well known in the art.

The manifold 170 also includes a plurality of injection ports 120 for injecting contrast medium into the contrast medium path, conduit 182, which is formed in part by the manifold. Typically, the injection ports 120 include luer lock connectors 186 for receiving and holding syringes filled with contrast medium.

The manifold 170 may also include a recess 190 configured to receive at least part of an air bubble detector. For example, the recess 190 may be formed to receive the sensor 134 discussed with respect to FIGS. 3 and 3A. The manifold 170 may also be configured with flattened sides 194 along the conduit 182 so as to facilitate mounting/coupling of the sensor to the manifold. By having the sensor 134 (FIG. 3) disposed in the recess or otherwise mounted along the conduit 182, the air bubble detector will be able to detect any air bubble introduced upstream from the manifold 170 and from the syringes and injection ports 120. Furthermore, the air bubble detector is able to detect aggregated bubbles which may develop by the combination of a number of smaller bubbles which may have been unseen by the surgical staff or believed to pose no threat individually.

FIG. 3C shows a minor variation of the manifold 170′. Rather than using a recess to receive the air bubble sensor 134, the air bubbler sensor is provided with a spring coupling 136 to connect the transmitter and receiver in a friction fit so that the sensor structure holds the components to the manifold 170′ adjacent the conduit. A sheath may be used to protect the sensor 134 from contamination, or the air bubble sensor may be resterilized after each use.

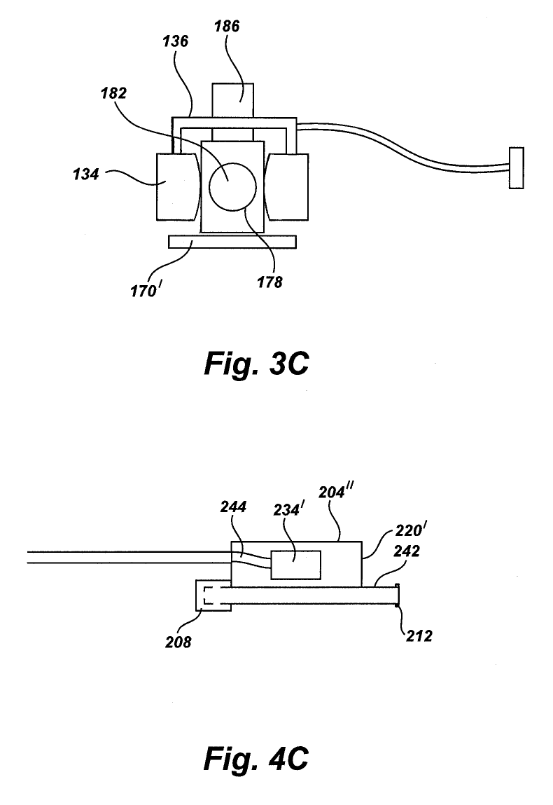

FIG. 4 shows a top, fragmented view of a contrast medium injection system 200 formed in accordance with the principles of the present invention. The system 200 includes the pump 108, the contrast medium infusion line 104, the manifold 112, syringes 116 and catheter 124 as used in the prior art. Disposed between the manifold 112 and the catheter 124 is an air bubble detector 204. The air bubble detector 204 is different than that discussed with respect to FIGS. 3 through 3B in that it lacks wire leads to a remote device.

The air bubble detector 204 preferably has a first connector 208 for attachment to the manifold 112, or to an intervening structure. Those of skill in the art will appreciate that pressure transducers can be attached to the manifold for ensuring that the contrast medium does not exceed certain pressure thresholds. The air bubble detector 204 also includes a second connector 212 for attachment to the catheter, etc. Thus the air bubble detector 204 is placed along the flow path of the contrast medium and is able to detect air bubbles from any source upstream. Typically, the connectors 208 and 212 will be luer lock connectors. However, other connectors could be used. The air bubble detector 204 may communicate wirelessly or may incorporate an alarm, etc.

FIG. 4A shows one embodiment of the air bubble detector 204. The detector 204 includes a housing 220 with connectors 208 and 212 configured to allow the air bubble detector to be placed along the fluid flow path. Typically the connectors 208 and 212 will be luer lock connectors (typically one male and one female), but other types of connectors may be used.

The house 220 holds a sensor 234 formed by a transmitter 234a and a receiver 234b. As was discussed previously, it will be appreciated that the transmitter 234a and receiver 234b may transmit and receive sound or light or other signals for determining the presence of air. For simplicity, the sensors will be discussed in the context of an ultrasonic sensor.

Power is supplied to the sensor 234 by a power source 238, such as a battery. The ultrasonic transmitter 234a sends acoustic waves through the conduit 242. The ultrasonic receiver 234b generates a signal in response to the acoustic waves detected. The signal may then be processed by a signal processor 246. The sensor results are then sent via a transmitter 250 to a remote monitor 254. The transmitter 250 may operate on radio frequency, via BLUETOOTH, or other wireless protocols.

Once the monitor 254 has received the information from the transmitter 250, the monitor will provide some indication whether there is an air bubble in the contrast medium. This can be done via a visual display 260, via a flashing light 264 and/or via an audible alarm 268. Input means 266, such as buttons, keys or dials, may be used to adjust the sensitivity of the sensor and/or alarms.

One advantage of the visual display 260 is that it could keep a cumulative count of bubbles detected. Thus, for example, the sensor 234 may not have detected a single air bubble which would cause concern. However, if it detected a number of small air bubbles, the monitor could display an approximate volume of air which had been injected. The physician could then determine if the risk of an air embolus was too great to proceed. Additionally, the monitor 254 could be disposed in communication with the pump 108 (FIG. 2) to suspend contrast medium injection if the total volume of air injected was above a predetermined threshold. Additionally, the air bubble detector 204 could be disposed in communication with a pressure sensor so that adjustments to volume calculations could be made depending on the pressure of the contrast medium at the time the bubble was detected. It will be appreciated that the amount of air in a bubble can vary greatly depending on the pressure.

Turning to FIG. 4B, the there is shown an alternate embodiment of an air bubble detector 204′ in accordance with the present invention. The air bubble detector 204′ includes connectors 208 and 212, a conduit 242 and housing 220 so that the air bubble detector forms part of the flow path. It also includes a sensor 234 with a transmitter portion 234a and a receiver portion 234b. It also may include a battery 238, a conduit 242 and circuitry for processing a signal generated by the sensor 234. Rather than transmitting the signals to a remote location, however, the air bubble detector 204′ is self contained. In other words, it includes a means for developing a human perceptible signal, such as a flashing light 270 and/or a speaker 274 for emitting an audible alarm.

While less sophisticated than the embodiments discussed in FIGS. 3 through 3A and 4A, the air bubble detector 204′ has the advantage of being generally less expensive, and minimizes potential interference with the angiography procedure. Once attached to the manifold 112, the air bubble detector 204′ provides virtually no additional impediment to the physician. However, it provides an important safety enhancement by detecting air bubbles which may have developed since or been unnoticed during inspection and priming of the system.

FIG. 4C shows yet another aspect of the invention. Ultrasonic bubble detection above has been described using a transmitting piezoelectric material and a receiving piezoelectric material in what may be described as a “pitch-catch” scenario. Ultrasonic bubble detection, however, does not necessarily require two separate piezoelectric pieces. An alternate method for conducting ultrasonic bubble detection is the use of a “pulse-echo” arrangement. In a pulse-echo scenario, a single piezoelectric element is used. The element produces an ultrasonic signal and then receives a reflected signal either from the bubble in the conduit, or from the opposing wall of the conduit. It will be appreciated that any of the sensors 134 or 234 or those discussed hereafter could use a pulse-echo configuration rather than a pitch-catch configuration.

In FIG. 4C, an air bubble detector 204″ includes a housing 220′ with a conduit 242 and connectors 208 and 212 so that the conduit can form part of the fluid path. The sensor 234′ is formed by a single piezoelectric element configured for a pulse-echo analysis of whether a bubble is disposed in the conduit 242. Wiring 244 is provided to send signals to and receive signals from the sensor 234′. The configuration shown in FIG. 4C is advantageous as the small number of parts makes the product less expensive to manufacture and more disposable. While the circuitry and alarms shown in FIG. 4B could be included in the housing, having the signals sent by wires to a separate processing/monitoring unit may reduce the cost of the sensor.

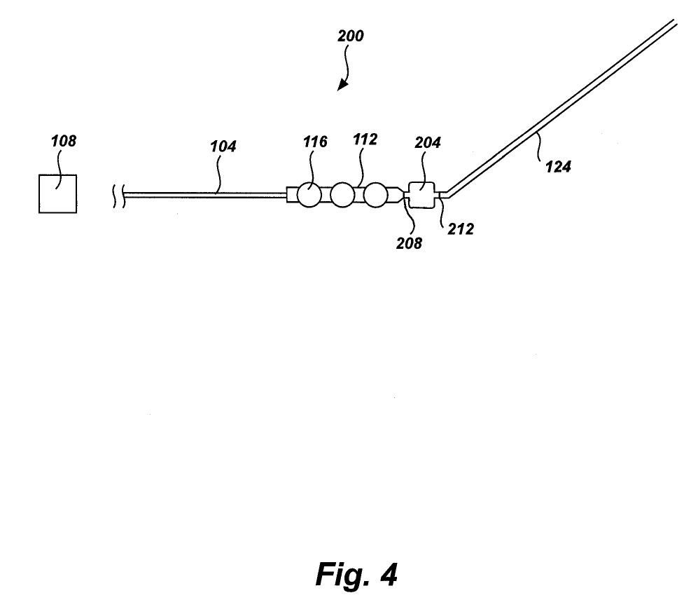

Turning now to FIG. 5, there is shown an alternate embodiment of an air bubble detection system, generally indicated at 300. The system 300 includes an air bubble sensor 304 with a transmitting portion 304a and a receiving portion 304b. The air bubble sensor 304 is configured to mount on the catheter 124. Typically, this could be done adjacent the luer lock connector 124b of the catheter 124. For example, the housing 308 which holds the sensor 304 could have a recess into which the top of the luer lock connector 124b nests. In the alternative, the housing 308 could have a hole through which the catheter 124 is advanced.

A pair of wires 312 extend from the sensor 304. The wires 312 carry activation signals to the sensor 304, such as by wire 312a, and carry signals generated by the sensor back to a monitor 316. It will be appreciated that the monitor 316 may have all of the features and components discussed with respect to monitor 254 shown in FIG. 4A. Preferably, the monitor can be placed outside of the sterile field 318, or otherwise protected to avoid the need to resterilize the monitor with each use. The wires 312 and the sensor 304, however, can be made as either a disposable item, or can be configured for resterilizing. Alternatively, the entire air bubble detector could be self contained such as the air bubble detector 204′ (FIG. 4B).

To facilitate either a disposable sensor 304 or a resterilzeable sensor, all of the processing circuitry, power and means for developing an alarm signal or other indication means can be disposed in the monitor 316. Thus, the monitor can be reused, while the sensor 304 can be discarded.

The configuration shown in FIG. 5 is highly advantageous because the determination of whether a bubble is present is made downstream of any point in which air could be introduced into the system. Even if air were to be drawn into the system via the luer lock connector 124b on the catheter 124, it will be detected by the sensor 304 and, if necessary, an alarm can be generated.

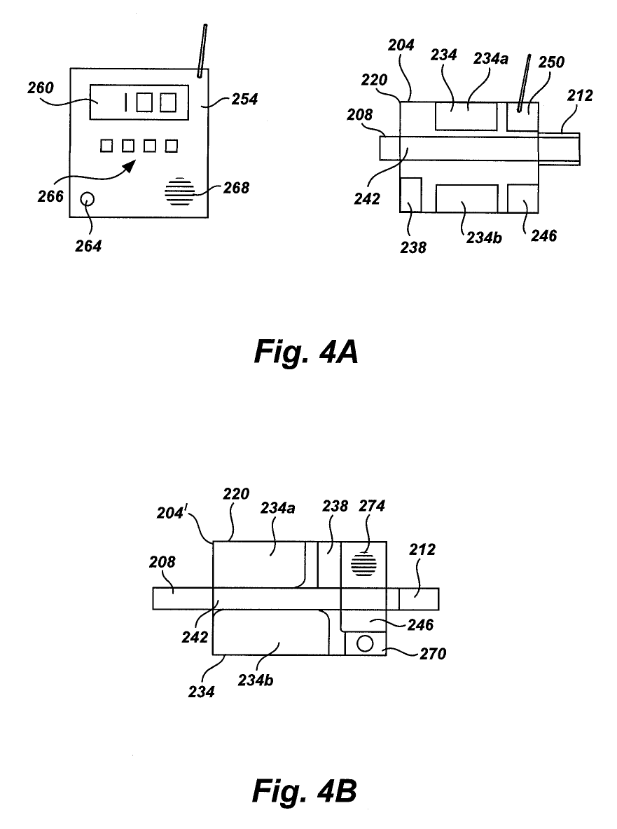

Turning now to FIG. 6, there is shown an alternate embodiment of air bubble detection system, generally indicated at 350, in accordance with the principles of the present invention. Rather than having a sensor mountable on the catheter, the luer lock connector 324 of the catheter has the sensor 334 built in. A pair of wires 328 extend from the sensor 334 to a monitor 316 which can be placed outside of the sterile field, if desired. It will be appreciated that the monitor 316 may have all of the features and components discussed with respect to monitor 254 shown in FIG. 4A. Thus, the catheter 124 comes with its own air bubble sensor 334 which is able to detect bubbles from any point of origin upstream from the catheter. The monitor 316 can be reused after a procedure and the catheter 124 discarded. In the alternative, the entire air bubble detector may be disposed in the catheter 124. It will be appreciated that most, if not all, of the sensors discussed herein may be integrated into a catheter 124 as is shown in FIG. 6.

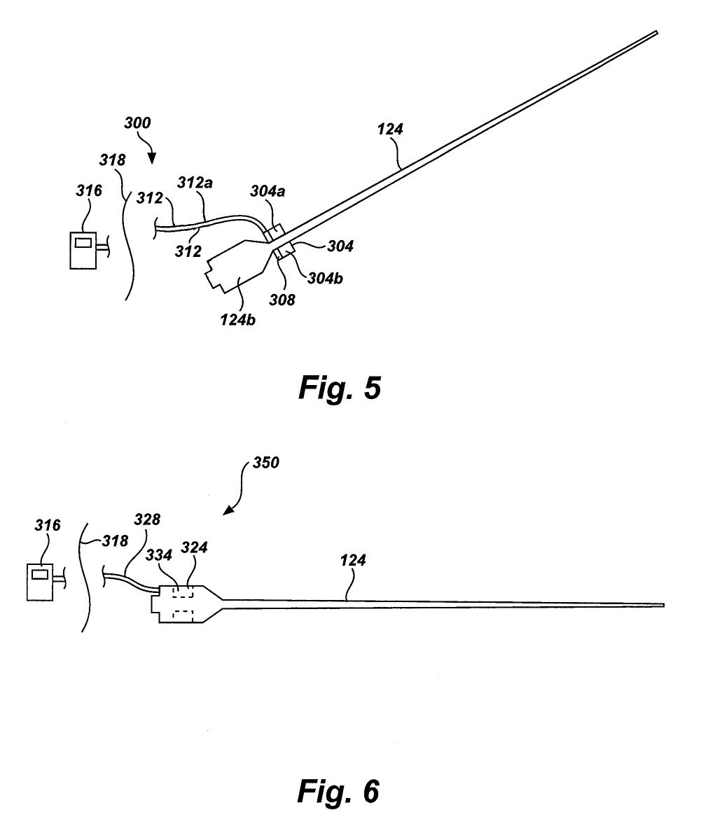

While the air bubble detectors described herein may be used in the more complex systems using a manifold, they may only be used in more simple procedures requiring only a single syringe. Thus, FIG. 7 shows a syringe 116 and catheter 124, with an air bubble detector 204 disposed between the two. If the syringe 116 has not been properly checked, or if the attachment 370 between the syringe and the air bubble detector 204 has drawn in air, the air bubble detector will detect the air and allow the physician to stop the procedure and to remedy the situation prior to recommencing the procedure. It will be appreciated that air bubble detector 204 can be made in accordance with any of the embodiments discussed above.

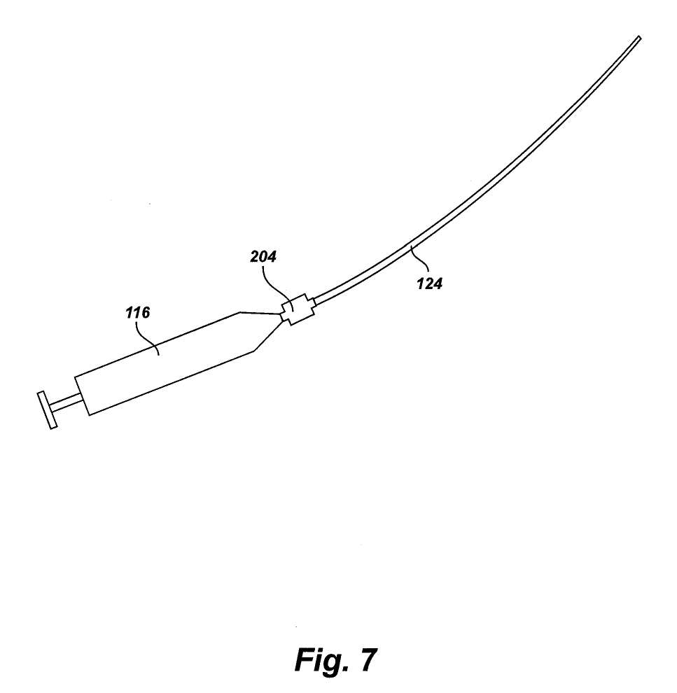

It will be appreciated in light of the present invention that at least a portion of the air bubble detector may be integrated into a manifold to reduce set-up time and decrease the number of connections through which a leak could occur. Thus, FIG. 8 shows a manifold, generally indicated at 400. The manifold 400 includes a housing 404 forming a conduit 406 with connectors 408 and 412 at either end. The manifold also includes injection ports 420 through which syringes, etc., can inject contrast medium or other fluid into the conduit.

The manifold also has an air bubble sensor 434 attached to or disposed in the housing 404. The air bubble sensor 434 may include a pair of piezoelectric elements, a single element, an optical bubble detector which interacts with a portion of the conduit 406 or other types of air bubble sensors. By integrating the manifold and the air bubble sensor less attachments are used, thus reducing the potential number of leaks.

The manifold 400 may carry only the air bubble sensor 434 or may include an entire air bubble detector, generally indicated at 440. Thus, the housing 404 may enclose or have attached thereto, processing circuitry 444, a battery 446 for powering the sensor 434, etc., The housing 404 may also enclose or have attached thereto alarm or indication means for generating a human perceivable signal, such as a light 448 and/or a speaker 450. A control switch 454 for turning the sensor 434 on or off, adjusting sensitivity of the sensor 434 and/or the volume of the alarm may also be used. A visual display 458 may also be provided. It will be appreciated that if the desire is to keep costs to a minimum, the manifold 400 may only include the air bubble sensor 434 and wiring 460 for connection to a remote monitor, etc., which would have the processing circuitry, power supply, etc.

Additionally, the manifold 400 may also include one or more other sensors, such as a pressure sensor, a flow rate sensor, or a cumulative dosage sensor, shown collectively at 470 and 474. Those skilled in the art will appreciate that such sensors can be implemented in a number of different ways. Additionally, the sensors 470, 474 may share the control circuitry 444 and battery 446, or may utilize wiring 460 to communicate with remote processors, etc.

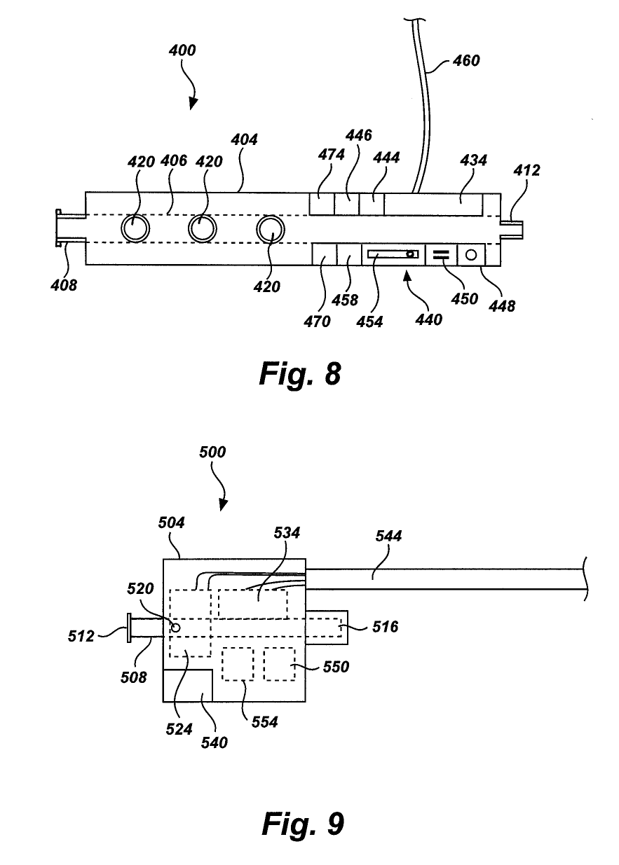

FIG. 9 shows a combined pressure sensor and air bubble detector in accordance with the principle of the present invention. The combined sensor, generally indicated at 500, includes a housing 504 with a conduit 508, having connectors 512 and 516 at the ends thereof for attachment along a fluid flow path in an injection system. The connectors 512 and 516 may be luer lock type connectors, or other connectors used for fluid injection streams.

The combined sensor 500 also includes a port 520 which provides access to the fluid for a pressure sensor 524 to determine the pressure of the fluid in the conduit 508. Disposed along the conduit 508 is also an air bubble sensor 534 for detecting the presence of air bubbles. The pressure sensor 524 and/or the air bubble sensor 534 may be powered by a battery 540 or other power source in the housing, or may be powered by wiring 544 which extends to an external monitor. Likewise, signal processing and the generation of alarms can be performed by sending signals to remote monitors, etc. via the wiring 554. In the alternative, a power supply 540, circuitry 550, and monitoring devices and alarms 554 such as those discussed with respect to FIG. 8. may be contained within the housing. Such elements are represented by boxes 550 and 554.

The combined sensor 500 shown in FIG. 9 is advantageous in that is provides for pressure monitoring and air bubble detection with the addition of only one small structure to the fluid injection system. Thus, the risk to the patient of excessive pressure of or an air embolus is virtually eliminated.

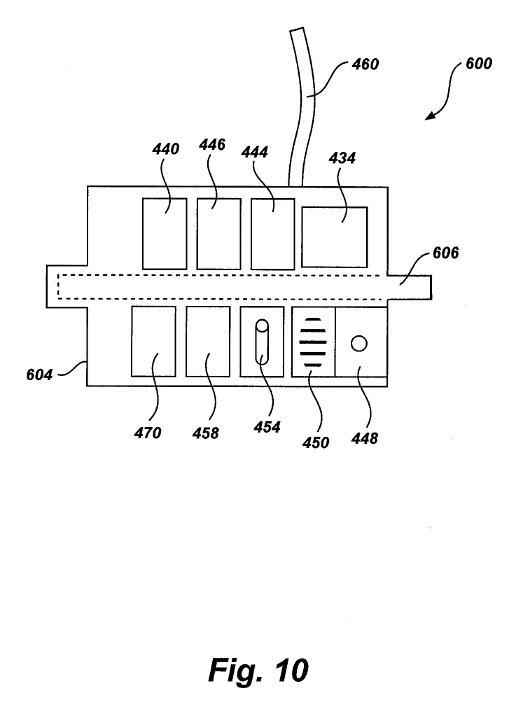

FIG. 10 shows a multifunction inline detector or sensor, generally indicated at 600 in accordance with the principles of the present invention. The multifunction inline sensor 600 preferably includes a air bubble sensor 434 for determining if there is an air bubble in the conduit 606 of the housing 604. The sensor 600 may include any or all of the sensors, switches, wiring and indication means, 440, 444, 446, 448, 450, 454, 458, 460, 470 and 474 discussed with respect to FIG. 8. Thus the multifunction inline sensor 600 may essentially be the manifold 400 of FIG. 8 without the injection ports 420. Thus the multifunction sensor 600 may be attached to a prior art manifold to provide some or all of the same functions as the manifold 400 in FIG. 8,

Those skilled in the art will appreciate numerous different ways for forming the various sensors. For example, a flow rate sensor could be formed by measuring the pressure change on the fluid as it passes through a constriction. Flow rate monitoring could also be determined more invasively, such as a sensor which extends into the fluid passing through the conduit. Cumulative dosage can be determined by knowing the volume, the flow rate and the time for which the fluid has been flowing.

Thus there is disclosed an improved method and apparatus for detecting air during an angiography or other medical procedure. Those skilled in the art will appreciate that numerous modifications could be made to the various embodiments disclosed herein without departing from the scope and spirit of the invention. The appended claims are intended to cover such modifications.

What is claimed is:

1. A fluid injection system comprising:
an infusion path configured for injecting fluid into a patient;
at least one injection port configured for injecting a fluid into the infusion path;
and
an air bubble detector having a sensor disposed distally from the at least one injection port.

2. The fluid injection system of claim 1, comprising a manifold defining a portion of the infusion path, the manifold having a plurality of injection ports, and wherein the air bubble detector sensor is disposed distally from the plurality of injection ports.

3. The fluid injection system of claim 2, wherein the manifold is configured for receiving the air bubble detector sensor.

4. The fluid injection system of claim 3, wherein the manifold has a conduit, and an outer wall disposed adjacent the conduit, at least a portion of the out wall having generally flat sides for coupling with the air bubble detector sensor.

5. The fluid injection system of claim 2, wherein the air bubble detector sensor is attached to the manifold.

6. The fluid injection system of claim 5, wherein the air bubble detector comprises a conduit and a sensor disposed adjacent the conduit, and wherein the conduit is attached to the manifold so as to be disposed distally from the manifold.

7. The fluid injection system of claim 1, wherein the infusion flow path comprises a catheter, and wherein the air bubble detector is configured for mounting on the catheter.

8. The fluid injection system of claim 1, wherein the infusion flow path comprises a catheter, and wherein the air bubble detector is formed as part of the catheter.

9. The fluid injection system of claim 1, wherein the air bubble detector further comprises a monitor having an indication means for generating an alarm when a bubble is detected.

10. The fluid injection system of claim 9, wherein the indication means is configured for being disposed outside of a sterile field and remote from the air bubble detector sensor.

11. The fluid injection system of claim 10, wherein the air bubble detector further comprises a transmitter for conveying signals from the sensor to the monitor.

12. The fluid injection system of claim 10, wherein the indication means is disposed distally of the injection port.

13. The fluid injection system of claim 9, wherein the air bubble detector comprises a housing and wherein the air bubble sensor and the indication means are disposed in the housing.

14. A fluid injection system comprising:
at least one syringe having contrast medium disposed therein;
at least one catheter disposed downstream of and in communication with the at least one syringe, so as to define a fluid flow path; and
an air bubble detector disposed along the fluid flow path downstream from the syringe

15. The fluid injection system of claim 14, wherein the syringe is attached to a manifold, and wherein the air bubble detector engages the manifold to determine whether air bubbles are present as fluid passes through the manifold.

16. The fluid injection system of claim 14, wherein the air bubble detector comprises an swept frequency ultrasonic air bubble sensor.

17. The fluid injection system of claim 14, wherein the air bubble detector comprising a housing having a conduit and an air bubble sensor for detecting air bubbles in fluid flowing through the conduit.

18. The fluid injection system of claim 14, wherein the air bubble detector comprises an alarm for indicating the detection of an air bubble.

19. The fluid injection system of claim 14, wherein the air bubble detector comprises an air bubble sensor disposed in a sheath.

20. A device for detecting air bubbles, the device comprising:
a housing comprising a conduit; and
an air bubble sensor attached to the housing for detecting an air bubble within the conduit.

21. The device according to claim 20, wherein the air bubble sensor is disposed inside the housing.

22. The device according to claim 20, further comprises luer connectors at either end of the conduit.

23. The device according to claim 20, further comprising wiring disposed in communication with the air bubble sensor for carrying signals to and from the air bubble sensor.

24. An air bubble detector comprising the device according to claim 23, and further comprising a monitor connectable to the wiring for processing signals from the air bubble sensor and for developing a human perceptible signal if an air bubble exceeding a desired threshold is detected.

25. The air bubble detector of claim 24, further comprising a control for regulating a fluid pump in response to detection of an air bubble.

26. The device according to claim 20, further comprising a transmitter in communication with the air bubble sensor for carrying signals to and from the air bubble sensor.

27. An air bubble detector comprising the device according to claim 26, and further comprising a monitor disposed in communication with a receiver for receiving and for processing signals from the air bubble sensor and for developing a human perceptible signal if an air bubble exceeding a desired threshold is detected.

28. The air bubble detector of claim 27, further comprising a control for regulating a fluid pump in response to detection of an air bubble.

29. The device of claim 20, further comprising a battery for powering the air bubble sensor.

30. The device of claim 20, further comprising circuitry for processing signals generated by the air bubble sensor.

31. The device of claim 20, further comprising an indication means for generating a human perceptible signal in response to detection of an air bubble.

32. The device of claim 20, further comprising a switch for adjusting sensitivity of the device.

33. The device according to claim 20, further comprising a pressure sensor.

34. The device according to claim 20, further comprising a flow rate sensor.

35. The device according to claim 20, wherein the housing comprises at least one fluid injection port for injecting fluid into the conduit.

36. The device according to claim 20, wherein the housing comprises a manifold.

37. The device according to claim 36, wherein the air bubble sensor is mounted on the exterior of the manifold.

38. The device according to claim 36, wherein the manifold has a recess for receiving the air bubble sensor.

39. The device according to claim 36, wherein the manifold has flattened walls for engaging the air bubble sensor.

40. The device according to claim 36, wherein the air bubble sensor is spring loaded to hold onto the manifold.

41. The device according to claim 20, wherein the air bubble sensor is disposed in a sheath.

42. A system for administering fluids comprising the device of claim 20, and further comprising a manifold attached at one end of the conduit and a catheter attached to an opposing end of the conduit.

43. The device according to claim 20, wherein the air bubble sensor comprises at least one piezoelectric element.

44. The device according to claim 43, wherein the air bubble sensor comprises means for sweeping the frequency of the at least one piezoelectric element.

45. A device for detecting air bubbles in a catheter, the device comprising:
an air bubble sensor; and
means for mounting the air bubble sensor on the catheter.

46. The device for detecting air bubbles of claim 45, wherein the air bubble sensor comprises at least one piezoelectric element and wherein the means for mounting the air bubble to the catheter comprises a housing.

47. The device for detecting air bubbles of claim 46, wherein the housing has a recess for receiving a portion of the catheter.

48. The device for detecting air bubbles of claim 46, wherein the housing has a hole formed therein for receiving the catheter.

49. The device for detecting air bubbles of claim 45, wherein the catheter has a connector and wherein the air bubble sensor is disposed in the connector.

50. An air bubble detection system comprising the device according to claim 49, and further comprising a monitor disposed in communication

51. The device according to claim 45, wherein the air bubble sensor comprises at least one piezoelectric element and means for sweeping the frequency of the at least one piezoelectric element.

52. A method for detecting air bubbles in system for injecting fluid into a human body, the method comprising;
selecting a system for injecting a fluid into a human body having a fluid flow path and at least one injection port for injecting fluid into the fluid flow path; and
disposing an air bubble sensor downstream from the at least one injection port.

53. The method according to claim 52, wherein the method comprises disposing a conduit having an air bubble sensor attached thereto along the fluid flow path.

54. The method according to claim 52, wherein the system for injecting a fluid into a human body includes a pump, and wherein the method comprises selectively deactivating the pump in response to a signal that an air bubble has been detected.

55. The method according to claim 52, wherein the air bubble sensor comprises at least one piezoelectric element and wherein the method comprises sweeping the frequency of the piezoelectric element.
